(19) United States
(12) Patent Application Publication
(10) Pub. No.: US 2006/0085080 A1
(43) Pub. Date: Apr. 20, 2006

(54) Medical device comprising a bio-compatible polymeric product with a layered structure

(75) Inventors: Klaus Bechgaard, Kobenhavn (DK); Tom Logstrup Andersen, Roskilde (DK); Aage Lystrup, Roskilde (DK); Povl Brondsted, Koge (DK); Sune Lund Sporring, Tureby (DK); Jes Bruun Lauritzen, Copenhagen (DK); Ole Olsen, Charlottenlund (DK)

Correspondence Address: GIFFORD, KRASS, GROH, SPRINKLE & CITKOWSKI, P.C, PO BOX 7021, TROY, MI, 48007-7021, US

(73) Assignee: Cartificial A/S, Kobenhavn o (DK)

(21) Appl. No.: 10/530,505

(22) PCT Filed: Oct. 10, 2003
(86) PCT No.: PCT/DK03/00686

§ 371 Date: Aug. 24, 2005

(30) Foreign Application Priority Data
Oct. 11, 2002 (DK) PA200201567

(51) Int. Cl. A61F 2/28; C08J 7/18

(52) U.S. Cl. 623/23.43; 623/23.51; 623/23.58; 623/926; 428/411.1; 264/101; 264/102; 264/241; 264/322; 264/258; 264/235; 427/488

(57) ABSTRACT

Disclosed is a method and a medical device comprising a bio-compatible polymeric product with a layered structure comprising at least one upper layer of a first polymeric component, a middle layer of a second polymeric component, and at least one lower layer of a third polymeric component, wherein the chain length of the first polymeric component and the third polymeric component is longer than the chain length of the second polymeric component. The medical device combines the features of strength, durability and bio-compatibility as well as it has resistance to tear and wear and has a good compressibility. A preferred design of the medical device is a cup produced from a care or film of LDPE surrounded by two layers of UHMWPE fabric. The medical device can be used as implants in mammals, especially as artificial cartilage within joints to secure mobility of the joint.

### FIELD OF INVENTION

The present invention relates to polymers and products produced by polymers. It discloses a method for enhancing the quality of polymer products, especially polymeric products that are to be exposed to pressure, impact, wear and tear. The polymers disclosed herein are particularly useful for cartilage substitution and for products to be utilised in medical devices. Especially the product can be used as an artificial joint spacer made to replace the missing cartilage, so the joint can stay mobile. All patent and non-patent references cited in the present application, are hereby incorporated by reference in their entirety.

### BACKGROUND OF INVENTION

Many prosthetic medical devices are implanted into load-bearing joints such as knees, hips, etc. As such, these prosthetic devices must be very strong and possess a high degree of wear resistance. Presently, the prosthetic medical device industry has utilised various metals and polymers and combinations thereof to fabricate prosthetic devices. Unfortunately, both metals and polymers have drawbacks. For example, metals such as stainless steel, tungsten and titanium, and alloys thereof, may succumb to the corrosive environment of the body and eventually begin to wear. Such wear may result in fine metallic particles being scraped away from the contact surface of the device and into surrounding tissue and bone which may potentially cause pathogenic problems. Polymers, such as polyethylene, polypropylene and nylons may also exhibit wear and may consequently produce particles which diffuse into tissue and bone. Both metallic and polymeric particles shed from these prosthetic medical devices are of concern because they may be inherently reactive with the tissue and bone they contact, thus possibly causing tissue degradation or necrosis.

Many medical devices are implanted into load-bearing joints such as knees, hips, etc, or utilised in the human body where mechanical function provide high strength or shape stability such as heart valves, breast prosthesis, stent, catheter, etc. As such, these medical devices must be very strong and possess a high degree of wear resistance. Prosthetic medical devices manufacturers constantly work toward developing better products by improving their physical properties. Improved wear resistance, for example, is a desirable quality to impart to a prosthetic medical device. Improving wear resistance without losing strength or causing oxidative degradation is a difficult balance to obtain.

Generally, joint damage, such as cartilage damage, is treated by replacing the joint with an artificial joint. However, serious complications are caused by the replacement of artificial joints, in particular a high occurrence rate of loosening problems resulting in breakage of the bones around the artificial joint. In the case of cartilage damage a repair with cartilage substitution placed into intact bones is to be preferred instead of replacing the entire joint.

Various methods have been devised attempting to reduce the wear rate of the load bearing prosthetic medical devices. For polymers, a common practice within the prosthetic medical device industry is to use cross-linked polymers and resins to form the medical device. Polymers are commonly cross-linked by chemical catalysis or irradiation exposure. Most cross-linking methodologies do result in greater wear resistance. However, indiscriminate or uncontrolled cross-linking may result in the formation of a weakened polymeric matrix, not capable of withstanding the enormous pressures placed on the devices in the patient resulting in degradative wear as described above.

One common practice within the medical device industry is to use cross-linked polymers and resins to form the medical device. “Cross-linked” polymers are defined as polymeric materials which have been subjected to chemical or radiation-initiated activation resulting in dendritic bond formation between and amongst individual polymeric chains yielding new intermolecular and intramolecular networks. These cross-linked networks within the polymer provide chemical and physical properties, which are usually different from the virgin polymer. Such properties include increased wear and creep resistance, durability, etc. Indiscriminate or uncontrolled cross-linking of the polymeric material comprising the medical device may result in improved wear resistance, but strength and other desirable properties may be sacrificed.

Ultrahigh molecular weight polyethylene (hereinafter referred to as ‘UHMW poly-ethylene’ or ‘UHMWPE’) is commonly used to make prosthetic joints such as artificial hip joints. In recent years, it has become increasingly apparent that tissue necrosis and interface osteolysis, in response to UHMW polyethylene wear debris, are one cause of the long-term loosening failure of prosthetic joints. For example, the process of wear of acetabular cups of UHMW polyethylene in artificial hip joints introduces many microscopic wear particles into the surrounding tissues. The reaction of the body to these particles includes inflammation and deterioration of the tissues, particularly the bone to which the prosthesis is anchored. Eventually, the prosthesis becomes painfully loose and must be revised. It is generally accepted by orthopaedic surgeons and biomaterials scientists that the reaction of tissue to wear debris is the chief cause of long-term failure of such prostheses.

U.S. application 20020007219 describes radiation and melt treated ultra high molecular weight polyethylene prosthetic devices. The reference describes a medical prosthesis for use within the body which is formed of radiation treated ultra high molecular weight polyethylene having substantially no detectable free radicals. Preferred prostheses exhibit reduced production of particles from the prosthesis during wear of the prosthesis, and are substantially oxidation resistant. Methods of manufacture of such devices and material used therein are also described.

U.S. application 20020037944 describes crosslinking of polyethylene for low wear using radiation and thermal treatments. Described are methods for enhancing the wear-resistance of polymers, the resulting polymers, and in vivo implants made from such polymers. One embodiment of the invention presents a method whereby a polymer is irradiated, preferably with gamma radiation, then thermally treated, such as by remelting or annealing. The resulting polymeric composition preferably has its most oxidized surface layer removed. Another embodiment of the invention presents a general method for optimizing the wear resistance and desirable physical and/or chemical properties of a polymer by cross-linking and thermally treating it. The resulting polymeric composition is wear-resistant and may be fabricated into an in vivo implant.

U.S. application 20010049401 describes chemically crosslinked ultrahigh molecular weight polyethylene for artificial human joints. Disclosed is a method for enhancing the wear-resistance of polymers by crosslinking them, especially before irradiation sterilization. In particular, the invention presents the use of chemically crosslinked ultrahigh molecular weight polyethylene in in vivo implants.

### SUMMARY OF INVENTION

A first aspect of the present invention relates to a medical device comprising a biocompatible polymeric product with a layered structure comprising at least one upper layer of a first polymeric component, a middle layer of a second polymeric component, and at least one lower layer of a third polymeric component, wherein the chain length of the first polymeric component and the third polymeric component are longer than the chain length of the second polymeric component.

The first and third polymeric component may be similar or the first and third polymeric component may be different from each other.

In a preferred embodiment the present invention provides methods of producing medical devices. In another preferred embodiment the present invention provides methods of producing polyethylene medical devices. Specifically, the invention provides a stratified polymeric structure to produce the medical devices having improved wear characteristics. At least three layers of polymers are stratified, and said stratified product may be pressed into shape.

By the term ‘medical device’ is to be understood any device which can be used shortly or more permanently in any process including a medical treatment of an individual.

The term ‘stratified’ is used synonymously with the terms ‘layered’ and ‘stacked’ and means that the product is made by three or more layers or strata of polymer material.

The characteristics of the medical product in the present invention is high tensile strength and improved wear resistance as well as capability of absorbing shocks, impacts and pressure load, due to the stratified structure of the device and cross-linked polymers within the device. Wear resistance can also mean wearability.

The term ‘layered structure’ especially describe features according to the method of formation of the polymer product of the devices, although a layered structure may be or may not be visible macroscopically or observable microscopically within the device produced by the polymeric components. The layered structure of a device may be realised by chemical analysis of the polymeric product and also by microscopic method comprising light microscopy, contrast microscopy or NMR microscopy. A surface layer of the medical device is optional. The surface layer of the device can only be detected by chemical analysis.

In a preferred embodiment a cartilage substitute is produced comprising the polymeric material, said cartilage substitute may substitute for damaged cartilage especially within joints with intact bones, where it is being capable of partly or completely fill the role of natural cartilage in the joint.

The present invention also provides for the fabrication of various types of prosthetic devices and other medical devices. While the invention is not limited to any particularly shaped medical and prosthetic device, the preferred shapes include acetabular cups, hip endo-prostheses, knees, ankles, shoulders, tibial and femoral joints, finger and thumb members, vertebra, elbows, foot and toe members and wrist members as well as breast prosthesis, heart valves, stents, and catheters.

In one embodiment of the invention, the polymeric materials produced in relatively thin layers are stacked, preferred is a combination where layers with different constitution are located against each other. Layers comprising polymeric materials of two or more constitutions are utilised. Preferably one layer is composed of one polymeric material. One polymeric material comprises long polymeric chains such as fibres, whereas the other polymeric material comprises short chain polymer material. The thickness of each polymer layer of the device is selected in accordance to the thickness of the device and the number of layers decided to utilise for the device, a preferred thickness of each polymer layer is between 0.1 mm and 10 mm, optional but also preferred is a device constructed of polymer layers wherein the long polymer fibre are comprised in thinner polymer layers than the layers of the short chain polymer material.

Medical devices manufactured from the stratified polymer layers of the invention may be moulded in one or more processes comprising heating, vacuuming and pressing. The stacked polymer layers are conducted to heat to combine the polymer layers, simultaneously or afterwards the polymer product is conducted to a press in vacuum where the overall shape of the medical device is controlled. Any surplus of polymer material may be cut away.

The medical devices can be produced with attachments, or one or more apertures to improve the functionality within the body of the individual receiving the medical devise or to fasten the device in said body.

Accordingly, another aspect of the invention is a method for producing a medical device of a polymeric product, said method comprising obtaining a number of at least three polymer layers, and positioning the polymer layers in a sandwich composition, and shaping the sandwich composition of polymer layers by heating said composition followed by pressing it into a mould, where the heating and pressing processes are conducted in vacuum, and providing the polymeric product in a desired shape.

In an embodiment the medical device is subjected to irradiation to cross-link the polymeric material, the radiation source can be, but is not limited to, high-energy electrons, gamma rays, light photons, microwaves, preferred is high-energy electrons and gamma rays, more preferred is high-energy electrons.

To reduce the friction between the medical polymeric device and its surroundings in the body, the shaped polymeric product may be subjected to surface coating, the surface coating may be any bio-compatible coating material capable of reducing friction. In a preferred embodiment the product is coated with 10-500 nanometer of polyvinylpyrrolidone (PVP) by a plasma polymerisation treatment. The surface coating increases the lifetime of the device by increasing lubricating properties ant thereby decrease the friction.

Definitions

LDPE=Low-density polyethylene

UHMWPE=Ultra high molecular weight polyethylene

The term ‘core’ is used to describe a layer of polymer; said layer is made of short chain polymer material. The short chain polymer material of the core is cast to the desired thickness. The core is primarily used to enhance the capability of the device to absorb shocks, impacts and pressure load, the core can also attach two layers of fabric to each other.

The term ‘fabric’ is used to describe a layer of polymer; said layer is made of long polymer chains, such as polymer fibre. The fibres are combined in a textile structure.

The term ‘fibre’ is used as a unit of molecules, where the fibre is of relatively short length, and further characterised by a high ratio of length to thickness or diameter.

The term ‘filament’ is used as a single textile element of small diameter and very long length considered as continuous.

The term ‘film’ is used to describe a layer of polymer; said layer is made of short chain polymer material. The short chain polymer material of the film is cast to the desired thickness. The film is primarily used to attach two layers of fabric to each other, but also provide important features to the device as described below.

The term ‘inlay’ is used to describe a smaller layer of polymer; said layer is made of short chain polymer material. The short chain polymer material of the inlay is cast to the desired shape and thickness. The inlay is primarily used to enhance the capability of the device to absorb shocks, impacts and pressure load in areas of the device subjected to higher degrees of pressure and shocks.

The term ‘implant’ is used for a device, which is inserted into the body in order to replace or substitute for a function, which has been lost or impaired.

By the term ‘medical device’ is to be understood any device which can be used shortly or more permanent in any process including a medical treatment of an individual. In particular the ‘medical device’ may be an implant, wherein the term implant refers to a device meant to being inserted into the human or animal body for a long period of time, for example several years. The term ‘strand’ is used as an assembly of parallel filaments simultaneously produced and lightly bonded.

### DESCRIPTION OF DRAWINGS

FIG. 1 illustrates one three-layered stratified structure of the medical devices comprising fabric-core or inlay-fabric.

FIG. 2 illustrates the three-layered stratified structure of FIG. 1 which is surface coated.

FIG. 3 illustrates one three-layered stratified structure of the medical devices comprising fabric-film-fabric.

FIG. 4 illustrates the three-layered stratified structure of FIG. 3 which is surface coated.

FIG. 5 illustrates one five-layered stratified structure of the medical devices comprising fabric-core or inlay-fabric-film-fabric.

FIG. 6 illustrates the five-layered stratified structure of FIG. 5 which is surface coated.

FIG. 7 illustrates one surface coated seven-layered stratified structure of the medical devices comprising fabric-film-fabric-core or inlay-fabric-film-fabric.

FIG. 8A illustrates the seven-layered stratified structure of FIG. 7 as well as a cup-shaped device produced from said layered structure (8B) and sealing of the rim (8C).

FIG. 9 illustrates a flow chart in the production of the device.

FIG. 10 illustrates a human hip joint in which one embodiment of a medical device according to the invention is located in situ.

### DETAILED DESCRIPTION OF THE INVENTION

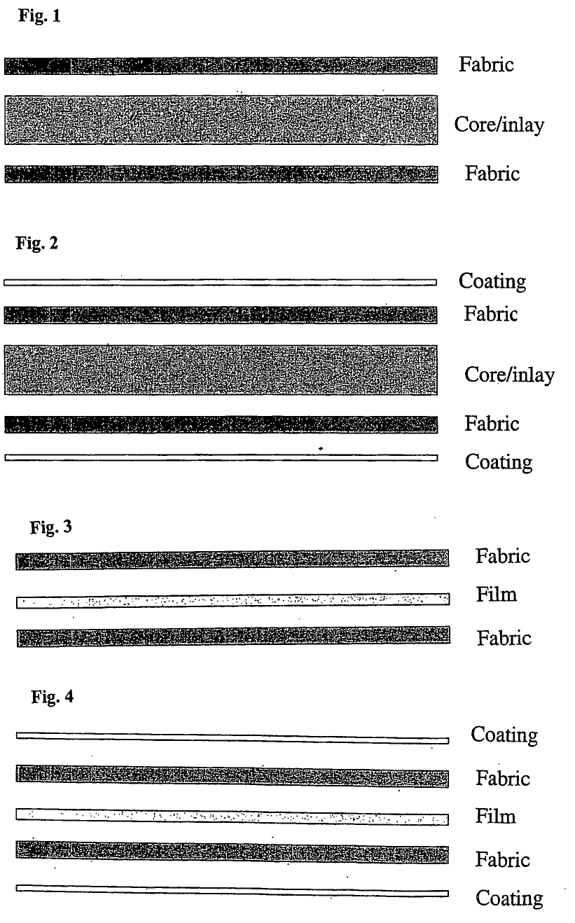

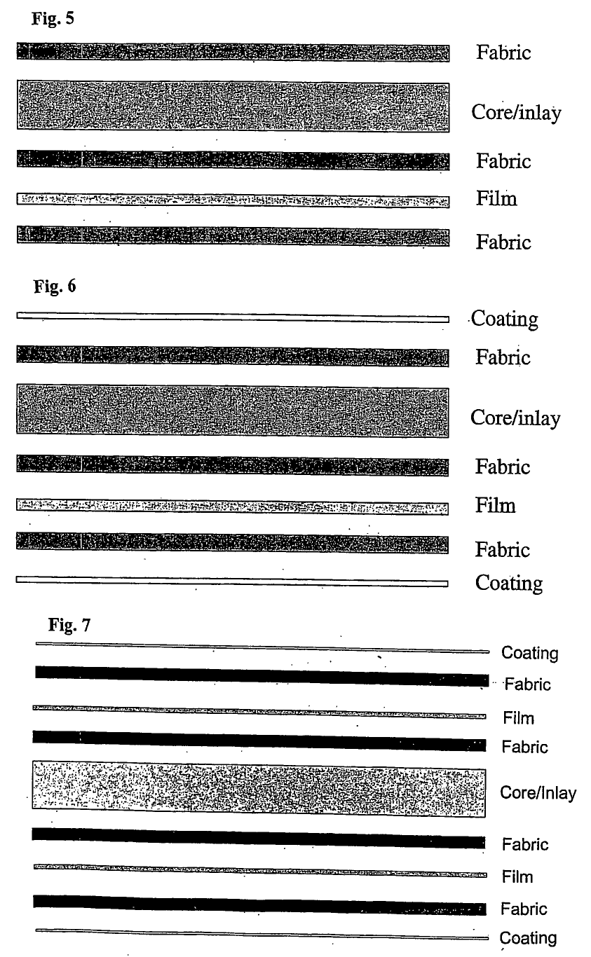

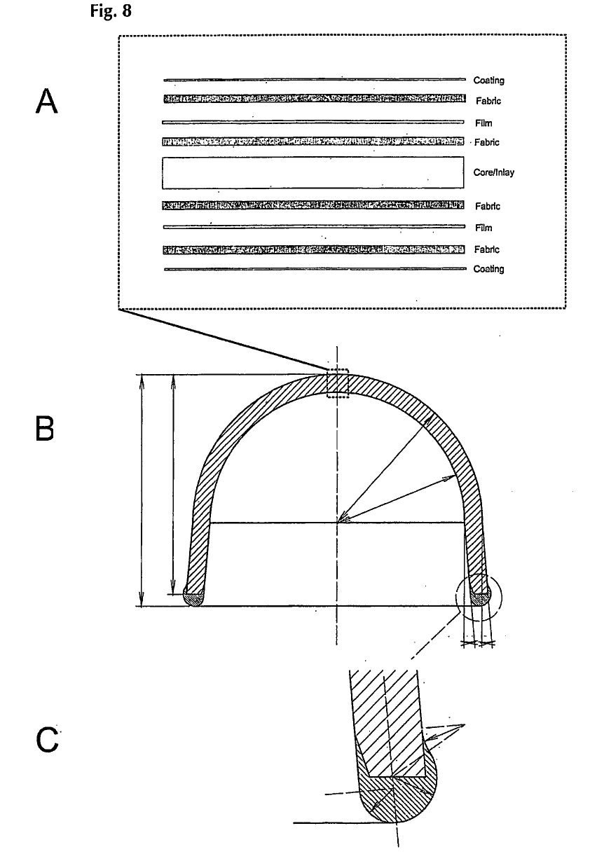

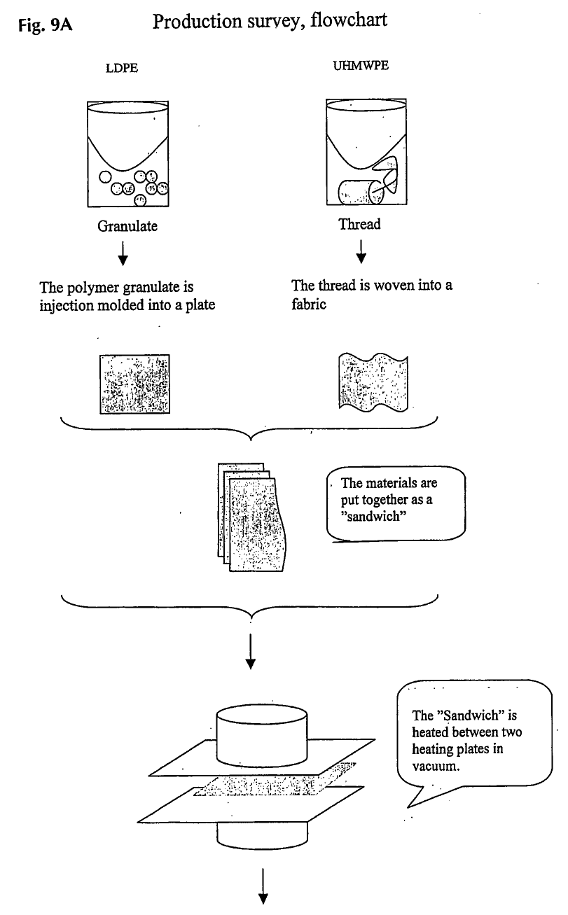

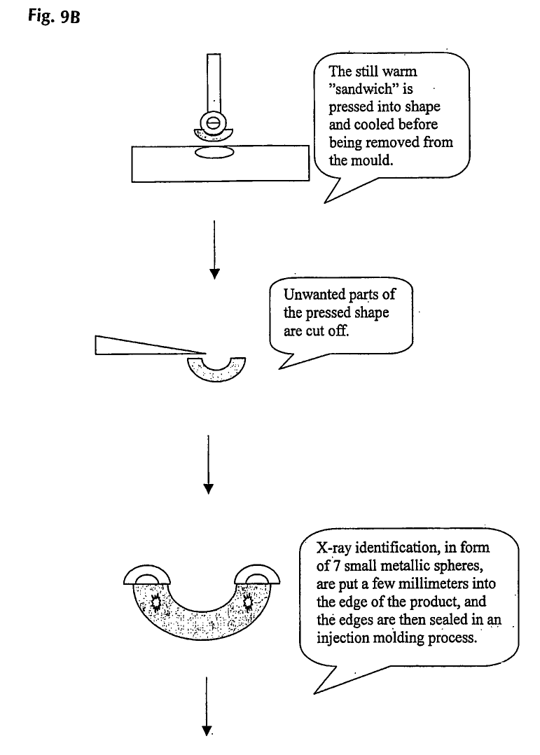

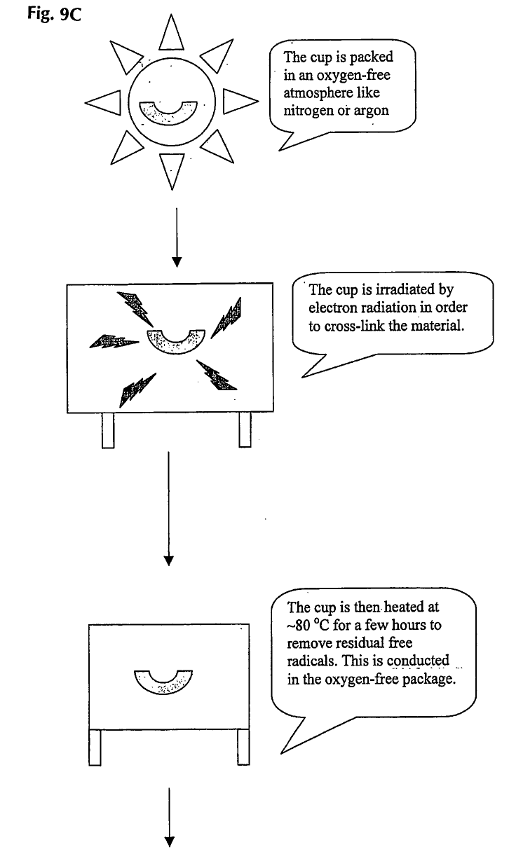

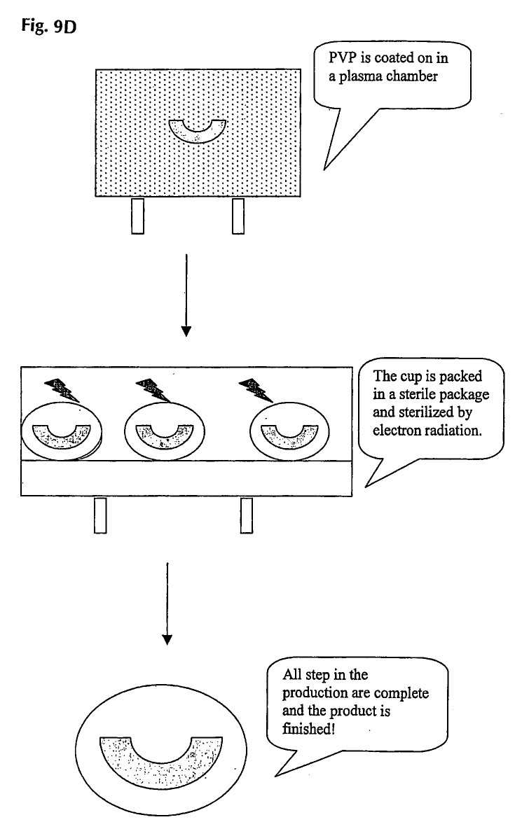

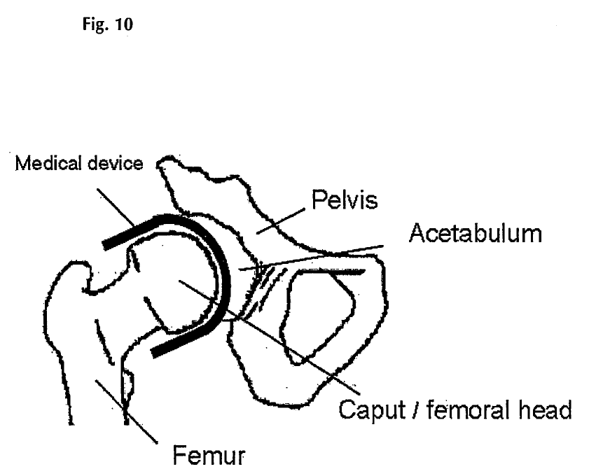

The present invention provides a desirable balance of improved wear resistance and high tensile strength and toughness in the polymeric compositions used for medical devices. It has been discovered that wear resistance can be improved without sacrificing other desirable properties such as toughness or strength by controlling the amount of different polymeric substrate comprising the prosthetic device. Referring to FIG. 1, the stratified structure of the medical devices is illustrated. The products of the invention has a high tensile strength and improved wear resistance as well as the capability to absorb shocks, impacts and pressure load, also it reduces the amount of tearing off.

The material for the medical devices is primary polymers, with at least one layer of a first polymeric component with high molecular weight, and at least a layer of a second polymeric component with low molecular weight. This combination of longer and shorter polymers provides the feature of the device comprising strength as measured by tear, tension and compression.

The main aspect of the invention is a medical device comprising a bio-compatible polymeric product with a layered structure comprising at least one upper layer of a first polymeric component, a middle layer of a second polymeric component, and at least one lower layer of a third polymeric component, wherein the chain length of the first polymeric component and the third polymeric component is longer than the chain length of the second polymeric component.

The essence of the present invention is the selection of the composition of the different polymer layers as well as the selection of the number of polymer layers comprising first, second and third polymeric components, as well as thickness of said polymer layers and also size and position of an optional layer of the second polymeric component.

Another embodiment of the present invention provides for layers of polyolefinic polymers and resins. Within the context of the present invention, a polymer is defined as an organic compound having repeating units of similar or different monomers. A resin is defined herein as a partially cured polymer having utility as a mouldable material suitable for curing into a solid article.

In an embodiment the polymers and resins of the polymer layers of the present invention may be polyolefinic polymers, polyethylene, polypropylene, polyacrylates, polystyrene, polytetrafluorethylene, polyvinylalcohol, polyethylene oxides, polyvinylpyrrolidon, polysilanes, polyurethanes, polyethers, polyamides, polyesters, polyalkyl acrylates, nylon, rubber and epoxy resins. It should be understood that the above list of polymers is not exhaustive, and other polymers may also be employed in the present invention. Preferred is polyethylene and polypropylene. Most preferred is polyethylene.

Preferably, the polymer materials of the first, second and/or third polymer layer may be from the group of polyethylenes or the group of polypropylenes such as polyethylene (PE), polypropylene (PP), high molecular weight polypropylene (HMWPP), high molecular weight polyethylene (HMWPE), ultra high molecular weight polyethylene (UHMWPE) and ultra high molecular weight polypropylene (UHMWPP), high density polyethylene (HDPE), low density polyethylene (LDPE), high density polypropylene (HDPP) and low density polypropylene (LDPP), ultra high density polyethylene (UHDPE), ultra high density polypropylene (UHDPP), cross-linked polyethylene, non-cross-linked polyethylene, cross-linked polypropylene, and non-cross-linked polypropylene. In this embodiment of the present invention, any combination of polymers listed above, or their equivalents, may be used.

First Polymeric and Third Polymeric Component

The polymers comprising the first polymeric component- and the third polymeric component are preferable above 100 monomer units, such as above 1,000 monomers units, for example above 10,000 monomer units, preferable above 20,000 monomer units, more preferable above 30,000 monomer units, further preferable above 40,000 monomer units, yet further preferable above 50,000 monomer units, most preferable above 60,000 monomer units.

The polymers of the upper and lower layer comprising first and third polymeric components of the present invention have preferably molecular weights ranging between 1,000 and 100,000,000 such as between 10,000 and 75,000,000, for example between 50,000 and 50,000,000, preferable between 75,000 and 25,000,000, more preferable between 100,000 and 1,000,000, further preferable between 200,000 and 800,000, yet further preferable between 300,000 and 700,000 most preferable between 400,000 and 600,000

In a preferred embodiment the polymers comprising the first polymeric component and the third polymeric component are comprises long polymer fibres, filaments or strands produced from the polymers presented above, preferred polymers to produce said fibre and filaments may be selected from the group of poly-ethylenes including, but not limited to, high molecular weight polyethylene (HMWPE), ultra high molecular weight polyethylene (UHMWPE), high density polyethylene (HDPE), ultra high density polyethylene (UHDPE), cross-linked polyethylene and non-cross-linked polyethylene. The most preferred polymer of the invention is fibre produced from UHMWPE. The polymers of the first polymeric component and the third polymeric product provide strength and wear resistance to the device.

A preferred polymer of the upper and lower layer of the invention is UHMWPE, and a preferred combination is UHMWPE and HDPE.

Additional components of the upper and lower layer polymeric material may be incorporated into the matrix in a braided, woven, spongy or spiral pattern, the fibres and filaments comprising the additional components having reinforcing properties. The fibres may be inorganic fibres such as carbide, nitride, boride, carbon and oxide fibres, or the reinforcement may be of organic origin such as Dacron.

The Second Polymeric Component

The middle layer comprising a second polymeric component can be constructed from short chain polymer material; the polymers may be selected from the polymers presented above. Short chain polymers may have less than about 100 units, such as less than about 90 units, for example less than about 80 units, preferable less than about 70 units, more preferable less than about 60 units, further preferable less than about 50 units, yet further preferable less than about 40 units, most preferable less than about 30 units. The short chain polymer material may not have cross links and only weak Van der Waals forces between chains, The molecular weight is preferably less than about 10,000, such as less than about 9,000, for example less than about 8,000, preferable less than about 7,000, more preferable less than about 6,000, further preferable less than about 5,000, yet further preferable less than about 4,000, most preferable less than about 3,000.

Preferred polymers to produce the middle layer constituting film, core and inlay polymer layers may be selected from the group of poly-ethylenes or from the group of polypropylenes including, but not limited to polyethylene (PE), polypropylene (PP), high molecular weight polyethylene (HMWPE), high molecular weight polypropylene (HMWPP), high density polyethylene (HDPE), high density polypropylene (HDPP), low density polyethylene (LDPE) and low density polypropylene (LDPP). Preferred is short chain polymer material such as LDPE and LDPP. Further preferred are polymers which are branched. Most preferred is short chain polymer material of polyethylene.

From the above mentioned first, second and third polymeric components different polymer layers comprising fabric, film, core and inlay are constructed. These polymer layers are further described below.

Fabric

From the longer polymers comprising the first and/or the third polymeric component as previously described a fabric may be constructed constituting the upper and lower layers of a medical device. Preferred is a fabric of UHMWPE fibre. The fabric corresponds to the first and/or third polymeric component as described elsewhere herein.

Within the fabric, the first polymeric component and the third polymeric component are preferably in the form of fibre. Methods of construction of fibres are known to persons skilled in the art. The polymers may be aligned and/or spun into fibre by gel spinning or filaments, which again may be spun into strands. From said fibres and/or filaments and/or strands the layers of polymeric materials may be manufactured.

The fabric may be produced into a suitable shape, said shape is preferably constructed by weave, knit, crochet, stitch, plait, interlace, intertwine, interlock, link or unite the fibre and/or filaments and/or strands in other ways such as non-woven techniques. Preferable the fabric is woven or knitted.

In an embodiment the fabric can be woven using different techniques, said techniques include but is not limited to cord woven, linen woven, mat woven, Celtic woven and twill woven. Persons skilled in the art know variations of these techniques, said variations is hereby incorporated.

According to an embodiment of the invention the polymer fibres are woven into a squared fabric comprising intercepts with angles of 90 degree. The dimension and weaving style of the fabric is optional, preferred is a binding style of 3:1 (twill). The fabric can if the thickness allows it be rolled into a roll, from which suitable pieces are detached before the stratified polymer product is constructed. Products which can be used comprises but is not limited to fabric of Dyneema® from DSM, Spectra® from Allied Signal Inc. Preferably the fabric is workable in the process of construction of the medical device as described elsewhere herein.

In another preferred embodiment the fibres, filaments or strands of the constitution described above are woven into the fabric in a shape suitable for the shape of the polymeric product. The shape of the fabric can be any possible shape including but not limited to round, oval, triangle, quadrangle, square, rectangular, pentagon, hexagonal etc. and may be symmetrical or asymmetrical in any direction. Preferred shapes of the fabric are quadrangle and round.

In one embodiment the fibres in each layer of the fabric are positioned over each other making a structure wherein the angles of the intersect are of 1 to 179 degree, such as in angles of 40 to 150 degree, for example such as in angles of 60 to 130 degree, such as in angles of 70 to 10 degree, for example such as in angles of 80 to 100 degree, such as in angles of about 90 degree. Most preferred is intersects of fibre and strands in angles of about 90 degree.

The thickness of the fabric is preferably determined by thickness as well as the number of fibres and/or filaments and/or strands and the distance between these fibres, filaments and strands in the fabric. The overall thickness of the fabric is preferably between 0.001 mm and 3 mm, preferred is between 0.01 mm and 2 mm, more preferred is between 0.02 mm and 1.5 mm, further preferred is between 0.03 mm and 1.0 mm, yet further preferred is between 0.04 mm and 0.08 mm, most preferred is between 0.05 mm and 0.06 mm.

In an embodiment the area weight of the fabric is preferred between about 10 g/M² and 500 g/M² preferred is an area weight of between about 50 g/M² and 300 g/M², more preferred is an area weight of between about 75 g/M² and 250 g/M², further preferred is an area weight of between about 100 g/M² and 200 g/M², yet more preferred is an area weight of between about 125 g/M² and 175 g/M², even more preferred is an area weight of between about 140 g/M² and 160 g/M², most preferred is an area weight of about 150 g/M².

The fibres, filaments and strands from which the fabric is produced according to the description herein, may have a fibre diameter preferably between 100 and 650 dtex. The fibre diameter of the warpyarn is preferably about 300-650 dtex, more preferably about 350-550 dtex, further preferably about 400-500 dtex, most preferably about 430-460 dtex. The weftyarn is preferably about 100-350 dtex, more preferably about 150-300 dtex, further preferably about 175-250 dtex, most preferably about 210-230 dtex.

The fabric need not be constructed of fibre or filaments or strands with equal thickness. A woven fabric where some of the strands have a larger thickness than the rest may be used. In this way e.g. every second, every third or more strands in between may have a larger thickness than the rest of the strands of the fabric.

The fabric described herein may also be constructed by strands of different polymers. Said different polymers may be selected among the polymers listed herein above. Two or more polymers maybe used in the construction of the fabric.

In an embodiment the thickness of the fabric may vary according to different thickness of the polymer strands as described above or different polymers utilised to construct the fabric. Also different numbers of strands pr cm may be used.

In an embodiment the surface dimension of one or more inner layers of fabric may be smaller than the total surface dimension of a medical device. Smaller layers of fabric may enclose inlays.

In another preferred embodiment the fabric has a high tensile strength and a high wear resistance. The degree of tensile strength is determined by the polymer utilised to produce the fibre and the thickness of the fibre. The tensile strength of the strand or fibre in a fabric is preferably above 1.0 GPa, such as above 1.2 GPa, preferable above 1.4 GPa, more preferable above 1.6 GPa, further preferable above 1.8 GPa, yet further preferable above 1.9 GPa, most preferable above 2.0 GPa.

In another embodiment the tensile strength of the strand or fibre in a fabric is preferably above 0.05 GPa, such as above 0.1 GPa, preferable above 0.3 GPa, more preferable above 0.5 GPa, further preferable above 0.7 GPa, yet further preferable above 0.8 GPa, most preferable above 0.9 GPa.

Although the term ‘fibre’ is used in the description of fabric comprising the first and third polymeric components, filaments and/or strands and/or other components comprising long chains of polymer units may be used instead of fibres.

The fabric constitute a reinforcement fabric or tissue of the device.

Film, Core and Inlay

The middle layer of the polymeric product comprises a second polymeric component. Said polymeric component may be any short chain polymer material or low density polymer material as described above. Also chopped strands of long chain polymer material such as fibre and/or filaments and/or strands may be utilised as short chain polymer materials. Preferred is when the chopped strands comprising short chain polymers are moulded into a matrix with low density polymer material or a polymer comprising the second polymeric component as described elsewhere herein.

By ‘chopped strands’ is meant shorter chains or strands cut from fibres and/or filaments and/or strands.

In an embodiment said middle layer comprising polymer layer comprises a film, a core or an inlay.

The polymer layers ‘film’, ‘core’ and ‘inlay’ may be produced of similar or substantially similar or different polymers. Preferred are polymer layers of film, core and inlay which are produced by similar polymers. Polymers suitable to be used are described above.

The differences of film, core and inlay may be the dimensions of the polymer layers. Said dimensions are determined according to the function of the polymer layers. The film, core and inlay may differ in thickness from each other, but may also have similar thickness, whereby film and core sometimes can substitute each other in the composition of the medical device.

The visual difference of film and core is preferably based on the thickness, where the film in general is thinner than the core. The main purpose of a film layer is to attach two layers of fabric to each other, and simultaneously provide the device with characteristics such as capability of absorbing shocks, impacts and pressure load.

The core may also attach fabrics to each other, and provide the same characteristics to the device as the film, but the core may be utilised in devises subjected to higher degree of impacts and pressure load than to devises comprising no core layer.

The difference of core and inlay may be based on the length and width of the polymer layers, the inlay may be smaller than a core. The function of an inlay is to absorb shocks and pressure in specific areas of a medical device. An inlay of one device may be larger than a core of another device.

In an embodiment said middle layer comprises a film or core or inlay. The film and core comprises the polymers described above, and may be constructed by melting said polymers. Mixtures of polymers may be used to construct the film, core or inlay. The melted polymeric mass may be formed according to any method possible, said methods are known to persons skilled in the art. Said methods comprises but are not limited to blow moulding, extruding, foil moulding, injection moulding, compression moulding, preferred is blow moulding. Preferred methods are moulding of the melted polymeric mass in small or large open moulds/vats or injection moulding, the thickness of the material is optional, but is chosen not to be changed followed solidification. Following solidification the solidified polymeric matrix can be cut or punched or stamped out to a suitable dimension.

The suitable dimension of the film may be determined in accordance to the scope of the application. The preferred application of the film is as a thin polymer layer between two layers of fabric, in this situation the size comprising length and width of the film is at least the length and width of the polymeric material used to produce a medical device, hereby the film may be squared, circular or any other dimension as any surplus of polymer material is removed following formation of the medical device.

In an embodiment the device is constructed from layers of fabric, film, core and/or inlay where said layers each has a dimension suitable to construct the device without any process of removing surplus of polymer layers. In this process the polymer layers of film, core and/or inlay may have dimensions smaller than the outermost layer of fabric. To adjust the size of the polymer layers to the form of the device to be produced, inner layers of fabric may be smaller than the outermost layer of fabric. The outermost layer of fabric which constitute the inner side of a medical device may also be smaller than the outermost layer of fabric which constitute the outer side of a medical device.

The suitable dimension of the core may also be determined in accordance to the scope of the application. The preferred application of the core is as a polymer layer between two layers of fabric, where the core fills in all the area comprising length and width between said two layers of fabric, in this situation the size of the core is at least the length and width of the polymeric material used to produce a medical device, hereby the core may be squared, circular or any other dimension as the surplus of polymer material is preferably removed following formation of the medical device.

The suitable dimension of the inlay may also be determined in accordance to the scope of the application. The preferred application of the inlay is as a polymer layer, which fills in part of the area between two layers of fabric or film; hereby the inlay may comprises any dimension appropriate for the purpose of the medical device. The inlay is moulded into the appropriate dimension or it is cut into the appropriate dimension.

The preferred thickness of the core and of the inlay is chosen in accordance with a reduction of said thickness in the construction of the device. During the pressing process the thickness of the core and the inlay may be reduced by up to 50%, as the short chain polymers of the inlay and/or of the core are pressed in between layers of fabric. By this pressing process the other dimensions comprising length in two dimensions of the core and inlay may increase as the thickness decreases.

Objects of non-polymeric material or objects of polymeric material different to the material which the core or inlay is made of, may be placed within core or inlay. Said objects may be but is not limited to metal globes or metal sheets. The objects may be incorporated in the inlay or core in the moulding process or may be placed in holes made in the inlay or core in the moulding process or made afterwards. An examples of objects in an inlay is metal globes in the inlay.

The difference of film and core has a fluid borderline, whereby the utility of film and core may be interchangeable. Also the difference of core and inlay has a fluid borderline, whereby the utility of core and inlay may be interchangeable

In an embodiment the film is prepared as described elsewhere, the film is preferably between 0.001 and 5 mm thick, such as between 0.01 and 5 mm, preferable between 0.1 and 4 mm, more preferable between 0.2 and 3 mm, further preferable between 0.3 and 2 mm, yet further preferable between 0.4 and 1.5 mm, most preferable between 0.5 and 1 mm.

The core or inlay which are also prepared as described elsewhere, is preferably between 0.1 and 30 mm thick, such as between 0.2 and 25 mm, preferable between 0.3 and 21 mm, more preferable between 0.4 and 17 mm, further preferable between 0.5 and 13 mm, yet further preferable between 0.6 and 10 mm, most preferable between 0.7 and 7 mm.

In an embodiment the surface dimension of one or more layers of film may be smaller than the total surface dimension of a medical device. Smaller layers of film may be used on one or more sides of smaller size fabric.

In a preferred embodiment the polymers as described above are of medical grade.

The film, core and inlay comprises short chain polymers. Examples of characteristics, properties and additives of said short chain polymers are shown in the following tables

| | Method | Unit | Value |
|---|---|---|---|
| Characteristics | | | |
| Melt index | ISO 1133 | G/10 min | 0.8 |
| Density | ISO 1183 | G/cm³ | 0.924 |
| Property | | | |
| Yield Stress | ISO R527 | Mpa | 12 |
| Tensile Strenght at break | ISO R527 | Mpa | 14 |
| Elongation at break | ISO R527 | % | 650 |
| Modulus of elasticity | ISO 527-2 | Mpa | 240 |
| Melting point | ISO 11357-3 | ° C. | 114 |
| Vicat temperature | ISO 306 | ° C. | 98 |

Mechanical properties measured on a moulded plaque.

More preferred examples of characteristics, properties and additives of said short chain polymers are shown in the following tables

| | Method | Unit | Value |
|---|---|---|---|
| Characteristics | | | |
| Melt index | ISO 1133 | G/10 min | 2.18 |
| Density | ISO 1183 | G/cm³ | 0.922 |
| Property | | | |
| Yield Stress | ISO R527 | Mpa | 11 |
| Tensile Strenght at break | ISO R527 | Mpa | 10 |
| Elongation at break | ISO R527 | % | 550 |
| Modulus of elasticity | ISO 5527 | Mpa | 210 |
| Melting point | ISO 11357-3 | ° C. | 112 |
| Melting point | ISO 11357-3 | ° C. | 94 |
| Shore hardness | ISO 868 | | 52 |

Mechanical properties measured on a moulded plaque.

Processing at an advised temperature of 150° C. to 180° C.

Additives may be used in the short chain polymer material, preferred is none slip agent and none anti-blocking additives.

An example of a short chain polymer material is Lacqtene® FE 8000 from Atofina.

Production of the Layered Polymeric Product

In one embodiment the polymeric product from which medical devices are constructed comprises polymer layers in a sandwich or laminated format with at least three polymer layers, where the polymer layers are fused together by a heating process. The middle or at least one inner layer differs from the two or more outer layers, hereby the polymer layers constitute a film, or a core or an inlay with at least one layer of fabric on each side. The fabric provides a high wear resistance and high tensile strength, while the inlay and core and to some degree also the film absorbs shocks.

In the production of a medical device, the different polymer layers as described above are laminated in accordance to the required characteristics of said medical device. In an embodiment the middle or at least one inner layer of the polymeric product constitute a core or a film, on each side of the core or film a fabric is positioned. In a preferred embodiment the polymeric product are composed of three layers where the fabrics at the different sides of the core have equal constitutions.

The layers of fabrics within a device can be different according to the polymers utilised to produce the fabrics or the fibres or strands within the fabrics may be different, or the fabrics are produced in different ways, also the fabrics can have different thickness. In a cup shaped device the outer part of the cup may comprise a thicker fabric than the inner part of the cup, hereby increasing the wear resistance of the outer part.

In another embodiment the polymeric product are composed of more than three layers, where, in between two fabrics a film or a core or an inlay are positioned. The individual layers of fabric may be substantially identical, identical or different in composition. Also the layers of film, core and inlay may be substantially identical, identical or different in composition.

In an embodiment the number of the layers core, film, inlay and fabrics differ across the polymeric product. The number of the layers can also vary in different areas of the polymeric product. The outermost layer of each side of the product must be fabric, and two layers of fabrics have a film or a core or an inlay in between. With varying number of layers across the polymeric product, also the thickness of the product varies. Some areas may contain an inlay other areas may be without said inlay.

In a further embodiment the outermost layer of the product can be a film. Said film is positioned next to a layer of fabric. Both sides of a product may have film layers as the outermost layers or only one side is a film layer. In case the medical device has more than two outer sides, one or more sides may be a film layer.

The polymeric product can also constitute two or more layers of fabrics on each side of a core or an inlay, and said two or more layers of fabrics may have a film of a polymer layer in between each fabric. Following heating of the layered polymeric product the film and/or core and/or inlay mechanically connect or bond together two layers of fabric.

Layers of film and/or core and/or inlay in a device may be different according to the polymer utilised to produce said layers. The polymers may be of different types, preferred polymers are mentioned herein above. Also the polymers may be mixtures of different types of polymers or mixtures of polymer chains of different length or both.

Film may have a higher adhesiveness than core and inlay.

The number of layers of fabric in a medical device is optional, as well as the number of layers of film and fabric and inlay can differ on each side of a core or of an inlay. The number of layers of fabric in a medical device is preferably between 1 and 100, such as between 2 and 50, for example between 2 and 40, preferable between 2 and 35, more preferable between 2 and 30, further preferable between 2 and 25, yet further preferable between 2 and 20, most preferable between 2 and 10.

Also the number of layers of film in a medical device is optional, the number of layers of film is preferably between 0 and 100, such as between 1 and 50, for example between 1 and 40, preferable between 1 and 35, more preferable between 1 and 30, further preferable between 1 and 25, yet further preferable between 1 and 20, most preferable between 1 and 10.

In addition the number of layers of core in a medical device is optional, the number of layers of core is preferably between 0 and 100, such as between 1 and 50, for example between 1 and 40, preferable between 1 and 35, more preferable between 1 and 30, further preferable between 1 and 25, yet further preferable between 1 and 20, most preferable between 1 and 10. The number of layers of film, inlay and fabric can be different at each side of a core.

The number of layers of inlays in a medical device is optional, the number of layers of inlay is preferably between 0 and 100, such as between 1 and 50, for example between 1 and 40, preferable between 1 and 35, more preferable between 1 and 30, further preferable between 1 and 25, yet further preferable between 1 and 20, most preferable between 1 and 10. The inlay can be positioned anywhere within the stratified polymer product between two layers of film or fabric. The inlay may be smaller than the entire area of the medical device, and the inlay may be located at any position within the medical device. Also the number of layers of film, core and fabric can be different at each side of an inlay.

Preferred layered compositions of the polymeric product of medical devices comprises but are not limited to the constitutions:
fabric-film-fabric.
fabric-core-fabric.
fabric-film-core-film-fabric
fabric-film-fabric-core-fabric-film-fabric.
fabric-film-fabric-film-fabric-core-fabric-film-fabric-film-fabric.
fabric-film-fabric-film-fabric-film-fabric-core-fabric-film-fabric-film-fabric-film-fabric.
fabric-film-fabric-film-fabric-film-fabric-film-fabric-core-fabric-film-fabric-film-fabric-film-fabric-film-fabric.
fabric-core-fabric-film-fabric-film-fabric.
fabric-film-fabric-core-fabric-film-fabric-core-fabric-film-fabric.
fabric-film-fabric-film-fabric-film-fabric-core-fabric-film-fabric.
fabric-film-inlay-film-fabric.
fabric-film-fabric-film-inlay-film-fabric-film-fabric.
fabric-film-inlay-film-fabric-film-fabric-film-fabric.
fabric-film-inlay-film-fabric-film-inlay-film-fabric.
fabric-film-inlay-film-fabric-film-fabric-film-inlay-film-fabric.
fabric-film-inlay-film-fabric-film-inlay-film-fabric-film-fabric.
fabric-film-inlay-film-fabric-film-inlay-film-fabric-film-inlay-film-fabric.
fabric-film-inlay-film-fabric-film-fabric-film-inlay-film-fabric-film-inlay-film-fabric.
fabric-film-inlay-film-fabric-film-fabric-film-fabric-film-inlay-film-fabric-film-inlay-film-fabric.
fabric-film-inlay-film-fabric-film-inlay-film-fabric-film-inlay-film-fabric-film-fabric.
fabric-film-fabric-inlay-fabric-film-fabric.
fabric-film-fabric-film-fabric-inlay-fabric-film-fabric-film-fabric.
fabric-film-fabric-inlay-fabric-film-fabric-film-fabric-film-fabric.
fabric-film-fabric-inlay-fabric-film-fabric-film-fabric-inlay-fabric-film-fabric.
fabric-film-fabric-inlay-fabric-film-fabric-film-fabric-film-fabric-inlay-fabric-film-fabric.
fabric-film-fabric-inlay-fabric-film-fabric-film-fabric-inlay-fabric-film-fabric-film-fabric.
fabric-film-fabric-inlay-fabric-film-fabric-film-fabric-inlay-fabric-film-fabric-film-fabric-inlay-fabric-film-fabric.
fabric-film-fabric-inlay-fabric-film-fabric-film-fabric-film-fabric-inlay-fabric-film-fabric-film-fabric-inlay-fabric-film-fabric.
fabric-film-fabric-inlay-fabric-film-fabric-film-fabric-film-fabric-film-fabric-inlay-fabric-film-fabric-film-fabric-inlay-fabric-film-fabric.
fabric-film-fabric-inlay-fabric-film-fabric-film-fabric-inlay-fabric-film-fabric-film-fabric-inlay-fabric-film-fabric-film-fabric.
film-fabric-film-fabric-film.
film-fabric-core-fabric-film.
film-fabric-film-core-film-fabric-film.
film-fabric-film-inlay-film-fabric-film.
fabric-film-fabric-film.
fabric-core-fabric-film.
fabric-film-core-film-fabric-film.
fabric-film-inlay-film-fabric-film.
fabric-film-fabric-inlay-fabric-film-fabric-film.

Wherein fabric comprises the first and/or third polymeric component as described elsewhere herein, and film, inlay and core comprise the second polymeric component as described elsewhere herein.

The layers of fabrics within a device may be similar or may be different in the construction. Also one or more layers of fabrics within a device may differ from the other layers of fabrics. Similar situations can be obtained regarding the film, inlay and core. Film, inlay and/or core of a single device may be different in construction.

By ‘different in construction’ is meant that the layers of interest can be produced by different materials or partly by different materials or the process of manufacture is different thus giving the layers different properties.

The constitutions of a product mentioned above may be surface coated by plasma polymerisation.

The polymeric material as described herein can also be used to cover prostheses of other materials, such as standard prostheses.

In another aspect of the invention the medical device comprises one or more layers of fabrics which may be surface coated by plasma polymerisation.

Features of the Product

The thickness of the polymeric product is determined by the number of polymer layers and the dimension of these layers in accordance to the requirements of the medical device. The total thickness of the polymeric product is preferably between 0.001 and 40 cm thick, such as between 0.005 and 30 cm, preferable between 0.01 and 20 cm, more preferable between 0.02 and 10 cm, further preferable between 0.03 and 8 cm, yet further preferable between 0.04 and 5 cm, most preferable between 0.05 and 2 cm.

In another embodiment the preferred thickness of a device is about 3 mm.

The surface area of a medical device may be between 1 cm² and 200 cm².

The surface dimension of a medical device comprising the polymeric layered structure as described herein may be between 0.01 to 40 cm according to length and width, such as between 0.05 to 35 cm, for example between 0.09 to 30 cm, preferable between 0.1 to 25 cm, more preferable between 0.2 to 23 cm, further preferable between 0.3 to 19 cm, yet further preferable between 0.4 to 17 cm, most preferable between 0.5 to 15 cm. Other preferred sizes of the surface dimension of a medical device may be between 0.5 to 8 cm according to length and width, such as between 0.5 to 7 cm, for example between 0.5 to 6 cm, preferable between 0.5 to 5 cm, more preferable between 0.5 to 4 cm, further preferable between 0.5 to 3 cm, yet further preferable between 0.5 to 2 cm, most preferable between 0.5 to 1 cm.

The surface dimension according to length and width of layers of fabric in a medical device as described herein may be substantially equal, equal or different from the surface dimension of the medical device. Preferred is a size where any surplus of fabric is removed following manufacture of the medical device.

To enclose one or more inlays with fabrics, the size of fabric according to the surface dimensions length and width may be the same as for the inlay, substantially the same as for the inlay or somewhat larger than the inlay. One or more inlays may be enclosed by two or more layers of fabric. Said layers of fabric may have surface dimensions adjusted to cover all the inlays, although the inlays may have distance between each other. Two or more inlays of a device may or may not be positioned in between the same two layers of fabric.

The surface dimension according to length and width of layers of film in a medical device as described herein may be substantially equal, equal or different from the surface dimension of the medical device. Preferred is a size where any surplus of fabric is removed followed manufacture of the medical device.

The surface dimension according to length and width of layers of core in a medical device as described herein may be substantially equal, equal or different from the surface dimension of the medical device. Preferred is a size substantially equal to the surface dimension of the medical device.

The surface dimension according to length and width of layers of inlay in a medical device as described herein may be substantially equal, equal or different from the surface dimension of the medical device. Preferred is a size where the inlay is smaller than the surface dimension of the manufactured medical device.

To increase the strength of the polymeric product, the layers of fabric may be turned according to each other, hereby the fibres of the different layers of fabric is positioned into different directions. The fabric may be turned between about 0 to about 90 degree, such as between 10 and 80 degree, preferred is between 20 and 70 degree, more preferred is between 30 and 60 degree, further preferred is between 38 and 52 degree, yet further preferred is between 42 and 48 degree, most preferred is about 45 degree in relation to the former and/or next layer of fabric.

Method for Preparation

Polymers may be prepared by methods known to the person skilled in the art. Chemical catalysis, thermal induction or photo induction are anecdotal non-limiting examples of methods of preparing the polymers.

The polymeric product is prepared according to the descriptions above by connecting the stacked polymer layers by heating. The heating temperature is selected below the melting temperature of the fibre crystallites in order not to loose the crystallinity of the polyethylene fibres and to a level where the fibres of the fabrics are not melted, destroyed or damaged, ie. below 250 degree Celsius, and above the melting temperature of the polyethylene plastomer, ie. in the range between 80 and 250 degree Celsius, such as between 90 and 240 degree Celsius, preferable between 100 and 230 degree Celsius, more preferable between 110 and 220 degree Celsius, further preferable between 120 and 210 degree Celsius, yet further preferable between 130 and 200 degree Celsius, most preferable between 140 and 190 degree Celsius.

In an embodiment the heating temperature is preferably between 90 and 200 degree Celsius, such as between 95 and 195 degree Celsius, preferable between 100 and 190 degree Celsius, more preferable between 105 and 185 degree Celsius, further preferable between 110 and 180 degree Celsius, yet further preferable between 115 and 175 degree Celsius, most preferable between 120 and 170 degree Celsius.

In a further embodiment the heating temperature is preferably between 90 and 180 degree Celsius, such as between 95 and 170 degree Celsius, preferable between 100 and 160 degree Celsius, more preferable between 105 and 155 degree Celsius, further preferable between 110 and 150 degree Celsius, yet further preferable between 115 and 145 degree Celsius, most preferable between 120 and 140 degree Celsius

When heated the short chain polymers of the core or the film or the inlay penetrate into the fibres or filaments or strands of the fabrics, and hereby mechanically connect or bond the polymer layers to each other. In a preferred embodiment the temperature is selected to a level where the main part of the fabrics is not melted, but a thin layer constituting a low number of polymer chains or fibres of the outer part of the outermost fabrics of the polymeric product is melted. The heating process may be provided in vacuum and under pressure.

In a further preferred embodiment the temperature is selected to a level where the performances of the reinforcement fibres are not damaged.

The polymer product comprising stratified polymer layers as described above, which has been subjected to the heating process, may be stored at room temperature until use. The polymeric product is preferable capable of being stored for long periods of time, such as several years. Storing is performed in dry conditions at room temperature and in darkness or at least without direct sunshine to the product. Dry conditions may be humidity of about 10-90%.

Shaping the Medical Device

In the heating process described previously or following a storage period of the fused polymer layers, the polymeric product comprising the polymer layers may be subjected to vacuum and may simultaneously be pushed or pressed into a mould to form the polymeric product. The vacuum process prevents the formation of bubbles and protects the polymer from oxidative degradation. Shaping can also be performed without vacuum but due to pressure optionally combined with heating the polymeric product.

The vacuum of the method described above may be below 500 mbar, preferable below 300 mbar, more preferable below 100 mbar, further preferable below 50 mbar, yet further preferable below 10 mbar, most preferable below 1 mbar.

To secure the desired shape of a device, the pressure of the device in the shaping process may be maintained until the polymer product is cooled preferable to room temperature. This cooling under pressure secures consolidation of the product.

The pressure in the process described above is a pressure high enough to press the product into a mould, the pressure may be a low pressure performed for a long period or a high pressure performed for a short period, or a pressure in between. Low pressure in this context is the pressure just enough to press the product into a mould.

The shape of the pressed polymer product may be any possible figure in each dimension where the shape may constitute a surface being flat, curved, waved, undulated, bent, bowed, crooked, while the overall shape of the device may be but is not limited to circular, oval, triangle, squared, rectangle, cubed, bowl, cup, crown, cap, basin, heart, egg, kidney, figure of eight, preferred shape is cup or hemispherical. The thickness of the device may also vary.

The polymer product pressed into a shape as described above, may be stored at room temperature for long periods of time, such as several years. In an embodiment the polymeric product may be produced with one or more apertures, holes, gaps, perforations or hollows. Said apertures etc. may constitute an improved attachment and/or optimise the function of the device. The improved attachment may be obtained without further processing as the apertures may constitute a shape of the device in a way that the device better fits into the location of the body. The apertures can also be utilised to fasten the device within the body.

Fastening methods are known to persons skilled in the art, and are hereby incorporated.

The apertures etc. may be created simultaneously with the shaping of the polymeric product, hereby the mould has points, tips or peaks, which create the apertures in the polymeric product. Another method of producing apertures etc. is to make a hole by a drill or another boring, cutting or pressing apparatus. Followed the formation of apertures etc. by drilling, cutting or pressing holes, the edge of the apertures on the polymeric device may be closed.

To the polymeric material may be attached a component, said component being polymeric or non-polymeric. The attachment may constitute part of a prosthesis or provide an anchorage point.

Collar

Following the shaping of the polymeric product as described elsewhere herein, surplus of polymeric material can be removed e.g. by cutting off. Cutting off the surplus of polymeric material leaves a polymeric product with right angle edges. These edges have to be rounded to secure no damage of the product is performed within the animal or human body when function as a medical device within said body.

In an embodiment the rim of the device may be treated to fix loose ends of fibre or strands. The rim may be closed by sewing or by fastening a polymer ring or a metal ring. When using a ring to close the rim of the device, said ring may be 0.55-5 mm thick, preferred is 3 mm.

In another embodiment a collar is placed on the medical device when surplus of material is removed. The collar can be moulded directly on the device e.g. by injection moulding.

The collar as described above can be of any material mentioned in the description of the first, second or third polymeric component. The collar material of the medical device can be the same material or a different material as actually used for the first, second or third polymeric component. Preferred is when the collar is of UHWMPE or LDPE. More preferred is collar of LDPE. Most preferred is when the collar is produced of the same polymeric component as actually used for the core or film, due to compatibility between the materials. Within the process the polymeric component of the collar may melt together with the polymeric component of the film and/or core.

In an embodiment a cup shaped medical device where the hat brim of surplus of material is removed a collar of LDPE is moulded directly on the cut edges by injection moulding.

Markers

In an embodiment markers are placed within the medical device. The markers can be used to visualise, trace or in other ways show the position of the medical device when inserted into a body. Visualisation can be performed by methods known to a person skilled in the art, which is hereby incorporated. One method is X-ray identification. The material of the markers can be any material, which can be placed within the polymeric material and can be detected from outside of the body.

In one embodiment the markers are contrast balls. Before closing the rim with a method described elsewhere, contrast balls are placed within the device. The contrast balls which can be any suitable colour such as but not limited to blue, red or green, can be placed in small holes drilled in the device. The drilled holes can be made at a right angle to the surface established when surplus of material is removed. Fastening a ring to the device or moulding a collar as mentioned above closes the holes. The number of contrast balls are optional, in a cup device 3-10 contrast balls may be utilised, 7 contrast balls is preferred.

In another embodiment the markers are made of metal. The shape of the markers is optional. Preferred are balls of metal. More preferred are markers of stainless steel or tantalum. Most preferred are balls of stainless steel or tantalum. The markers are placed within holes of the medical device drilled from the cut edge appearing when surplus of material is removed. The number of markers is optional. The number of markers is preferably 1-10, such as 2, e.g. 3, such as 4, e.g. 5, such as 6, e.g. 7, such as 8, e.g. 9, such as 10. The placing of the markers may be optional. Preferably the markers are placed asymmetrically around the cut edge of the medical device. This asymmetric placement ensures the possibility to measure if the medical device changes position when implanted in the body. In a cup-shaped medical device the asymmetric placement of the markers is preferable an asymmetric placement according to the circle comprising the cut edge of the medical device, hereby it can be visualised within the body whether the cup rotates. The cut edge is closed with a method described elsewhere herein.

In a further embodiment the markers are small pieces of the marking material. The small pieces are placed within the core or inlay when these are moulded.

In another embodiment the markers are formed as threads, and are placed within the core, inlay, film or fabric when these are produced. Threads of markers can also be placed between the fabric and any of the core, inlay or film when the medical device is constructed from said components.

Finishing Treatment of a Medical Device

The present invention in particular relates to material formulations intended to meet the specifications of durability, bio-compatibility, and strength. These properties are obtainable by treating polymer materials, such as polyethylene, polypropylene or polyvinylpyrrolidone or combinations and co-polymers thereof as well as precursor materials for polymerisation, with high-energy electrons, gamma rays, photons, microwaves, ion implantation, plasma treatment, annealing, thermal radiation or another radiation to obtain ideal durability and bio-compatibility of the new, modified material. Treatment of the above-mentioned materials with radiation leads to cross-linking of polymers and thereby generating new, modified materials. Preferably, the polymer material is a cross-linked polyethylene or polypropylene material. More preferably the polymer material is a cross-linked polyethylene material.

The properties of the materials to be obtained by the cross-linking process are preferably resistant to tear and wear; and have good compressibility.

The medical devicemay be packed in a pouch, which is suitable for irradiation. Preferred are pouches of aluminium, more preferred are laminated pouches of PE, aluminium and PET, where PE (polyethylene) comprises the inner of the pouches and PET (polyethylene-terephthalat) comprises the outer of the pouches.

A pouch with a medical devicemay be filled with nitrogen before it is made airtight. The medical devices are then subjected to irradiation to cross-link the polymeric material and sterilise the medical device.

In order to increase stability of the medical polymeric device the polymers of the shaped polymer product may be subjected to further treatment, such as cross linking. In a preferred embodiment the cross linking treatment is conducted in order to cross link only a fraction of cross linkable polymers in the product. Accordingly, the products may be cross-linked by radiation, the cross-linking of the polymers may also be done by other methods known to the person skilled in the art. Said radiation may be but is not limited to high-energy electrons, gamma rays, photons, and microwaves. Cross binding the polymers improve the strength of the product. A preferred radiation process is cross-linking of fibres using treatment with accelerated electrons. As the cross-linking process takes place in the amorphous polyethylene regions, the optimal dose will depend on the fraction of amorphous polyethylene in the final device. The optimal radiation dose is preferably close to the gelation dose of polyethylene and thus lie between 10^{-10,000} kGy (0.1 and 100 Mrad), preferred is between 10 and 300 kGy, most preferred is 200 kGy.

The radiation can be performed in one uninterrupted treatment, where the complete dose of radiation is given to the material. The radiation process may also be performed in pulsing or interrupting treatments, where the total dose of radiation is given in 2-15 shorter with an interval of 1 to 60 minute. Preferred is 25 kGy given eight times with 10 min interruption between each radiation treatment (total 200 kGy). More preferred are two times 25 kGy interrupted by 1 to 60 minutes and repeated 4 times with 10 hours to 1 day of interruption.

The radiation may be performed for the entire product or device or only part of the product or device is radiated by using a shield or screen between the irradiation source.

When using radiation, the radiation process described may be followed by annealing. The purpose of annealing is to eliminate long living free radicals by a heat treatment of 80° C. for about 1-12 hours in vacuum. More preferred is 70-85° C. for about 16-24 hours in an inert atmosphere. Preferred is when the inert atmosphere is Nitrogen.

Typically, a device is prepared by a process comprising the following steps:
The device is formed under vacuum by pressing the laminated polymers in a mould of specified dimensions. The polymer is chosen from the above mentioned polymers.
After hardening the material as formed, or after swelling in a suitable solvent, the device may be subjected to high-energy electrons, gamma rays or another radiation in order to create cross-linking which will modify the mechanical properties of the material to meet the preferred specifications.
Finally, after removal of the swelling solvent, the surface of the material may be treated to achieve good surface properties as described elsewhere.

The medical devices may be subjected to annealing when they are irradiated. Annealing is performed in an oven at about 80° C. for a few hours to remove residual free radicals. Or annealing is performed as described elsewhere herein.

Surface Coating

The surface of the device can subsequently be treated to modify surface properties such as wetting ability and/or biocompatibility. This surface treatment can be performed by plasma treatment, chemical grafting or by a combination of plasma polymerisation and chemical grafting. The material contacting with the biological surfaces may be smooth, biocompatible, preferably self-lubricating, and it should be wear-resistant so that particles generated due to wear are avoided in that this could otherwise result in foreign body reactions and cause further trouble to the function of the part of organism where the medical device is located.

Furthermore, the surface material should preferably be a material or a combination of materials having self-repairing properties so that fissures, cracks or other ruptures on the surface do not exceed uncontrollable levels. However, the surface material is preferably continuous with the material of the rest of the device, e.g. the material may gradually merge into the material of the fabric, film or core of the device. In this context continuous means that the surface material cannot be pulled away from the material beneath.

The surface of the material may be chemically treated so as to soften, rigidify or lubricate the surface of the device or parts thereof. The surface of the material may be coated so that the coating confers these properties, or may be treated so as to chemically alter the surface of the device so as to confer any of these properties. Alternatively, certain polymer surfaces may be modified by means of thermal or photolytic energy.

Without being bound by theory it is also believed that a wetted surface reduces the risk of having the immune system recognising the device when implanted, which would otherwise lead to adverse effects of the device.

In one embodiment the surface of the devicemay be coated by a plasma polymerisation, using low-power plasma equipment. The monomers used for the plasma polymerisation are any monomer forming a hydrophilic polymer by plasma polymerisation. Preferred are monomers forming polyvinylpyrrolidone and poly-ethylene-glycol like polymers, most preferred is 1-vinyl-2-pyrrolidinone.

The surface coating performed as described above has a thickness of 1 to 700 nm, such as between 10 and 500 nm, preferable between 20 and 400 nm, more preferable between 30 and 300 nm, further preferable between 40 and 200 nm, yet further preferable between 50 and 100 nm, most preferable between 60 and 90 nm.

In another embodiment the surface coating performed as described above has a thickness of 1 nm to 5,000 nm, such as between 5 and 2,500 nm, preferable between 10 and 1000 nm, more preferable between 30 and 500 nm, further preferable between 40 and 400 nm, yet further preferable between 45 and 300 nm, most preferable between 50 and 250 nm.

Plasma is ionised gas. In an artificial plasma to be used for plasma treatment and plasma polymerisation, the concentration of ionised species is preferably 0.1-10 ppm. Two phases exists in artificial plasma: A gas-phase comprising an energi corresponding to the surrounding temperature, usually room temperature. In a plasma-phase ions and electrons have an energi at approximately 2-10 eV.

The artificial plasma may be established by exposing a gas with electric field. The pressure of the gas is preferably 0.01-1 mbar. The electric voltage utilised is dependent of different features such as the pressure, the composition of the gas, electrode configuration, the size of the polymerisation chamber, and frequencies of the electricity. The voltage is typically 200-10,000V.

In a preferred embodiment of the plasma polymerisation 1-vinyl-2-pyrrolidinone (VP)may be polymerised to polyvinylpyrrolidone (PVP) in a plasma with low energi. The plasma functions as an initiator for the polymerisation by formation of radicals in the surface of the element to be coated. From the radicals the polymerisation process takes place where monomers of VP polymerise to PVP. A low energy is necessary not to destroy the monomer VP in the gas-phase as well as the polymerised PVP. In a preferred embodiment the energy is 0.1-1 W/IL.

In the plasma polymerisation treatment a carrier gas is used, preferred is an inert gas, such as argon or helium.

The chamber for performing the plasma treatment is constructed to perform a homogeneous surface coating of the device by the plasma polymerisation process.

The surface coated polymeric product is preferably sterilised by radiation or by heating. The radiation can be but is not limited to high-energy electrons, gamma rays, photons, microwaves.

The polymeric product may by cross-linked and sterilised simultaneously by treating with ionizing radiation or by heating. Preferred is cross-linking by radiation.

Mechanical Properties

The structure of the material of a device may comprise a layered or laminated structure, a core of one material or one or more interposed layers with different properties enabling an overall function of the devise suitable for providing a spacer function and/or to exert pressure distribution of joints and/or to provide at least part of the sliding/rotating movement of joints by internal movement of the device, or relevant part of the device. However, it is preferred that the material itself does not comprise interposed layers resulting in sliding between the layers and thereby tear on the mating surfaces within the device. Accordingly, the body of the device should be one continuous solid or semi-solid material.

Mechanical properties for certain relevant polymers are described by Szycher (Szycher, M. (editor), sponsored by SPE, Society of Plastics Engineers, Inc. Biocompatible Polymers, Metals, and Composites, pp. 725-727, 757-61).

Mechanical properties of polymers are controlled by the elastic parameters, the three moduli: elastic, shear, and compressive moduli. These parameters are theoretically interrelated. A modulus is the ratio between the applied stress and the corresponding deformation. The reciprocals of the moduli are called compliances. The three elastic moduli have the dimension: force per unit area, (N/m² or Pa). Polymers are not normally ideal elastic bodies, but under load they show (time dependant) viscoelastic properties. By taking the load into consideration, the properties should be viewed according to this dilemma. Also, ideal elastic properties and ultimate properties are influenced by the viscoelastic properties.

Ultimate tensile strength is a measure of the stress required to cause the material to rupture in tension. Ultimate elongation is the percent stretch of the material before it ruptures in tension. Elongation (%) is measured as

$$\text{Elongation (percent)} = \frac{{S}_{B} - {S}_{o}}{{S}_{o}} \times 100$$

where S_{B}=observed distance between bench marks of the stretched specimen at rupture, and S_{O}=the original distance between bench marks.

TABLE 1
Elastic parameters and their definitions

| Elementary mode of | | |
|---|---|---|
| deformation | Elastic parameter | Symbol |
| Isotropic (hydrostatic) | Bulk modulus | K |
| compression | bulk compliance or com- | κ |
| | pressibility | (κ = 1/K) |
| Simple shear | Shear modulus or rigidity | G |
| | Shear compliance | J (J = 1/G) |
| Uniaxial extension | Tensile modulus or | E |
| | Young's modulus | |
| | Tensile compliance | S (S = 1/E) |
| Any | Poisson ratio | ν |

| Symbol | Definition |
|---|---|
| K | $\frac{Hydrostatic Pressure}{\begin{matrix}Volume change per \\ unit volume\end{matrix}} = \frac{p}{ΔV \text{/} {V}_{0}} = \frac{{pV}_{0}}{ΔV}$ |
| κ (κ = 1/K) | reciprocal of foregoing |
| G | $\frac{Shear force per unit area}{\begin{matrix}Shear per unit distance \\ between shearing surfaces\end{matrix}} = \frac{F \text{/} A}{tan γ} = \frac{τ}{tan γ} = \frac{τ}{γ}$ |
| J (J = 1/G) | reciprocal of foregoing |
| E | $\begin{matrix}Force per unit \\ \frac{cross \text{-} sectional area}{Strain per length}\end{matrix} = \frac{F \text{/} A}{ln ( L \text{/} {L}_{0} )} = \frac{σ}{ɛ} = \frac{F \text{/} A}{ΔL \text{/} {L}_{0}}$ |
| S (S = 1/E) | reciprocal of foregoing (strain/stress) |
| ν | $\begin{matrix}Change in width \\ \frac{per unit width}{\begin{matrix}Change in length \\ per unit length\end{matrix}}\end{matrix} = \frac{lateral contraction}{axis strain}$ |

Examples of ranges of the mechanical properties of the device are mentioned below. However, it should be contemplated that not all of the following characteristics may be fulfilled by the material of the prosthetic device since, as explained above, the numerous properties of the material are theoretically interrelated. Accordingly, conflict in fulfilling all parameters within the stated ranges may occur.

In one embodiment, the prosthetic device according to the invention is a device wherein the material of the device or at least the part of the device which exerts the pressure distribution and/or the part which exerts the sliding/rotating movement in the joint when the joint is loaded has/have one or more of the following propeties (under biological conditions (37° C., physiological salinity)): A compressive modulus (K) of at least 2000 MPa, a shear modulus (G) of at least 1 MPa and an elastic module (E) of at least 10 MPa.

Furthermore, certain requirements to the material under stress with forces that ultimately leads to disintegration can be expressed. Based on the elasticity parameters for the material, the properties of the material with respect to pressure, elongation, torsion and displacement in the range where the material responds elastic can be estimated. The ultimate limits should preferably be within ±20% of the range of elastic response. As a consequence thereof, the limits for the ultimate properties (ultimate compression strength, tensile strength, torsional strength, shearing strength) can be derived. Furthermore, the material should have an “ultimate percentage elongation” of at least 20%.

The materials according to the invention may be a “quasi elastic” material. Y. Shikinami and H. Kawarada, Biomaterials 19, 1998, pp. 617-635, discuss that many materials of biological origin, has a J-form in a stress vs. strain curve, whereas may synthetic materials has an S-form.

Preferably, the critical surface tension (γ_{c}) values should be within the “zone of bio-compatibility” corresponding to the range of about 20-30 dynes/cm (as defined by Lelah M. D., Cooper, S. L., Polyurethanes in Medicine-CRC Press, Inc. Boca Raton, Fla., pp. 59-42 and 92-93).

Additives

A device constructed from the polymeric product may comprise biologically active additives. Medication or biological active substances can be used as additive to the device to facilitate healing, minimise destruction or with other therapeutic goals, such as pain relief, anti-inflammation, oncology treatments, stimulation of bone growth, and/or anti-infectious agents. Also, biological osteogenic or chondrogenic, chondral inductive, and/or chondral conductive materials may be added to the device. In particular patents suffering from osteoporosis or other bone degenerating conditions may benefit from having devices comprising osteogenic inductive materials implanted.

The medication or biological active substances can be used as additive to the device to facilitate cell growth, such as osteocytes; osteoblasts, chondrocytes, chondroblasts, mesenchymal cells. Cartilage inducing factor may for example be the factors described in U.S. Pat. No. 4,774,322 and U.S. Pat. No. 4,843,063.

In another preferred embodiment, additives such as lubricants, dyes, stabilizers and other process enhancing compounds are incorporated into the polymeric mixture. Such compounds may not necessarily enhance the strength or structural integrity of the final polymeric matrix, but do aid in the manufacturing process or enhance the overall appearance of the finished article. Examples of these compounds may be long chain fatty acids and their salts, organic and inorganic coloring agents, free radical inhibitors, pH buffering agents and other materials known to enhance processing of polymers within the polymer industry.

In another preferred embodiment of the present invention, solid materials may be incorporated into the polymer or resin mixtures. Such solid materials may be, for example, chopped carbon or glass fiber or nanotubes, carbon black, graphite powder, talc, mica, polyamide fiber and other fillers commonly used in the polymer industry. As is known in the polymer industry, such fillers may be advantageously added to a polymer matrix for the purposes of enhancing strength, durability, bulk density, machineablity of the resulting polymeric article. Of, course the above list is not exhaustive and other uses of the fillers may also be contemplated.

Devices

One preferred device produced of the polymeric product described herein may be a substitution for cartilage. Said cartilage substitution may replace damaged cartilage between intact bones, or it may be part of a medical prosthesis comprising cartilage substitution.

A device produced of the polymeric product itself can be used as a growth medium and/or network for the natural or artificial cells, such as chondrocytes.

A device made from the polymeric product described above is capable of being formed to suit into parts of the organism as described elsewhere herein. Especially the device is suitable to be used in animals, such as mammals and human beings, preferred is human beings. The animals, to which the medical device may be utilised, may be selected from the group of mammals, such as but not limited to horses, dogs, cats, cows and monkeys.

In one embodiment the device is especially constructed to be utilised to support, hold, sustain, bear, carry, replace or displace any constitution within the mammalian body, which comprises high shape stability and good wear resistance.

The polymeric product is adapted not to interfere with intra-articular or other components when the device is in the body of a human.

The polymeric product as medical device may be but is not limited to be used as joint spacer implant in joints of knees, hip, shoulders, fingers, wrist, elbow, spine, neck, loin, toes and ankles. Especially the devices are used in diseased patients with osteoarthritic degeneration of joints. The implants with a smooth articulating surface oppose the diseased and degenerated cartilage joint facet, which is expected to lead to reduced force and stresses and improved mobility in the joint with consequent reduced pain and improved functional capacity of that joint.

The medical device as described herein may be produced in a number of sizes corresponding to the natural variety of the bones within the joint where it is intended to be used as well as to the differences in bone size due to the age or size of individuals.

Moreover, non-interference of the intra-articular components may be achieved by a hole which runs through the body of the device; that is to say the device may comprise a hole through which intra-articular components may pass. When loading the device, the slits may serve to pass intra-articular components through the body of the device. The slits in this embodiment run from the periphery of the body of the device to the hole through which the intra-articular components pass after the device is implanted or loaded.

Typically, and to at least some extent, the device is adapted in its structure and/or material composition to alleviate conditions associated with worn cartilage by providing a spacer function and/or to exert pressure distribution in the joint when the joint is loaded and/or to provide at least part of the sliding/rotating movement of the joint by internal movement of at least part of the device.

It is also an object of the present invention to provide a method for non-invasive locking of a device within a joint. In addition, the method is independent of use of cement or bony ingrowth of the device.

The device may completely or substantially completely surround an intra-articular component or other components of the organism.

A device made from the polymeric product described above is capable of being formed to suit any joint cavity of animals or human beings, therefore the device may for example be formed to fit into any one of the following joints: Hip joint, knee joint, ankle joints, shoulder joint, elbow joints, wrist, fingers, spinal column joints, such as for substituting intervertebral discs, and the jaw joint.

The medical device may constitute the surface of a prosthetic device. It may be the entire surface or part of the surface of a prosthetic device Also the device may constitute a complete or part of a hip endo-prosthesis, or it may be a breast prosthesis, a stent, a catheter, a heart valve or cartilage substitution.

Generally, the invention comprises the polymeric product as described above from which different medical devices may be manufactured, also the method of producing said polymeric product and medical devices is enclosed within the invention. Enclosed are methods of producing a polymeric product and medical devices as described above, as well as any combination of the features described for said polymeric product and said medical devices.

Another aspect of the invention is a method for producing a polymeric product, said method comprising obtaining a number of at least three polymer layers, and positioning the polymer layers in a sandwich composition, forming the sandwich composition of polymer layers by heating said composition followed by pressing it into a mould, where the heating and pressing processes are conducted in vacuum, and providing the polymeric product in a desired shape.

In the method for producing a polymeric product wherein the polymeric product is as described above, at least three polymer layers is utilised, these polymer layers constitute a core with at least one layer of fabric on each side, where the core differs in constitution from the fabrics, preferred is the method for producing a polymeric product where the fabrics at the different sides of the core have equal constitutions.

The method for producing a polymeric product comprise two or more layers of fabrics, where said two or more layers of fabrics have a film of a polymer layer in between each fabric.

In the method for producing a polymeric product the core and the film have similar composition except for the thickness of the polymer layer. The thickness of the polymer layers is as described above, in a preferred embodiment the film is between 0.01 and 2 mm thick, and the core is between 0.1 and 10 mm thick. In an embodiment the method for producing a polymeric product comprises fabric, film and core where the structure of the fabric are composed of long polymer fibre, and the core and film are composed of short chain polymers. These polymer fibres can be selected among polyethylene (PE), polypropylene (PP) and polyvinylpyrrolidone (PVP). Most preferable is polyethylene (PE). The long polymer fibres are ultra high molecule weight polyethylene (UHMWPE) fibre and the short chain polymers may branched.

In an embodiment the method for producing a polymeric product comprises fabric which is manufactured, e.g., woven, into a shape or form suitable for the shape of the polymeric product. Said fabric consist of UHMWPE fibres in which the intersects are positioned as formerly described, preferably in angles of about 90 degree.

In an embodiment the method for producing a polymeric product comprises fabric which has high tensile strength and high wear resistance, and a core which absorbs shocks, pushes and strokes.

The method for producing a polymeric product comprises arranging the polymer layers in the order of fabric, film and core in accordance to the description above. The most preferred constitutions are listed above. The polymer layers are heated, and under vacuum the polymeric product is pressed in to a mould. The device, which is formed, is treated by ionising radiation, to further cross bind the polymers and thereby improve the strength of the product. The product is further subjected to annealing to ensure all linking has appeared.

In an embodiment the method for producing a polymeric product comprises surface coating of the annealed polymeric product and further the polymeric product is sterilised by ionising radiation or by heating.

In another embodiment the method for producing a polymeric product comprises annealing the polymeric product before it is subjected to surface coating.

In a further embodiment the method for producing a polymeric product comprises simultaneously cross-linking and sterilisation of the polymeric product by treating with ionising radiation or by heating.

In an embodiment the method for producing a polymeric product comprises surface coating of the polymeric product, as formerly described.

In a preferred embodiment the method for producing a polymeric product comprises production of the polymeric product where the shape and size of the polymeric product can be any possible to produce by pressing into to a mould, said mould forming a polymeric product which can be flat or round or in between and where the three-dimensional shape can be any possible forming by pressing into a mould.

The polymeric product can be utilised to produce a prosthetic device comprising polymer layers, the order of the polymer layers, and the method of production of the polymeric product as described above.

Preferred is a method of producing a prosthetic device of three polymer layers, which constitute a core with at least one layer of fabric on each side. Another preferred constitution is a core which at each side has two layers of fabric with a film in between. A further preferred constitution is a film between two layers of fabric.

In a preferable embodiment of the method the prosthetic device are produced from polymer layers composed of a polymer selected among polyethylene (PE), polypropylene (PP) and polyvinylpyrrolidone (PVP). Most preferable is a prosthetic device wherein the polymer layers are composed of polyethylene (PE).

In a further preferable embodiment of the method the prosthetic device are composed of fabrics of long polymer fibre, which preferable are ultra high molecule weight polyethylene (UHMWPE) fibre or other polyethylene fibre as previously described, whereas the core and the film are composed of short chain polymers, the short chain polymers may be branched.

The fabric is of medical grade and is woven into a shape suitable for the shape of the polymeric product. The shaping and physical characteristics is determined by the arrangement of the UHMWPE fibres, said fibres can have intersects in angles αs described formerly.

In a preferred embodiment the prosthetic device has a high tensile strength and a high wear resistance due to the properties of the fabrics, whereas the core absorbs shocks, pushes and strokes.

The polymeric constitution of the prosthetic device is obtained in accordance with the details given above where the polymer layers are heated, subjected to vacuum and pressed into shape in a mould, and further treated as described above.

### EXAMPLES

### Example 1

Artificial Cartilage Cup

The artificial cartilage cup is an artificial joint spacer made to replace the missing or damaged cartilage so the joint can stay mobile.

The cup is based on a sandwich construction with a LDPE core reinforced on both sides with UHMWPE fiber fabric.

At the edge metal markers makes it possible to trace the cup when implanted.

The round LDPE collar of the cup makes a cup without sharp edges and captures the metal markers.

Finally a crosslinking of the polymer improves the performance of the LDPE core.

The production process includes the following steps:

Injection Moulding of Base LDPE Disk

The LDPE disk is made of pellets/granulates in the injection moulding process. The disk is approximately five mm. thick and 134 mm in diameter. One standard disk size will later be formed to different size of cups.

Pressure Consolidation with UHMWPE Fiber Fabric

Two pieces of 20×20 cm UHMWPE fiber fabric are placed on each side of the disk and the sandwich are pressed to form a cup with a surplus material like an irregular hat brim.

Different cup sizes are produced and identified by an individual number.

Shaping the Cup by Cutting off Excess Material

Cutting off the hat brim leaves a cup with right angle edges.

Drilling of Cup Holes and Mounting of Metal Markers

Metal markers in the cup make tracing the cup in the body possible. For the first test production, the markers are tantalum balls, and for later production, the markers will be stainless steel balls.

Injection Moulding of LDPE-Collar on Cup

The metal markers are fixed, and LDPE-collar covers the right angle edges.

Packaging with Nitrogen Gas

In a packing machine, the cup is packed in an aluminum pouch with nitrogen gas, and the pouch is sealed to prevent oxygen from being in contact with the cup. Oxygen will hinder the later crosslinking process, as it reacts with the free radicals. The aluminium pouch is put in a shipment box, ready for sending to the crosslinking plant.

Crosslinking and Sterilization

The cup goes to the irradiation plant, is irradiated and returns to the production area. The irradiation forms free radicals. The free radicals are very reactive places in the polymer material, which react to form crosslinking in the polymer. The irradiation dose is about 200 kGy.

Annealing

Just after the irradiation process, there are still free radicals. In the annealing process, the free radicals form crosslinking. The annealing process is a heating at approximately 75° C., which speeds up the crosslinking reaction without softening the cup. The process is slowly running even at room temperature, but might take about one month. The temperature must be at a relatively low level in order to avoid softening and deforming of the cup.

Final Packing, Releasing and Storing

The cup is packed in inner box, labelled and instructions for use are supplied. The product is released after a quality check, and stored at the subcontractor.

### Example 2

The cup-shaped medical device constructed as a three layered device comprising fabric-film-fabric was tested to assess wear properties using a machine intended to simulate the tribological conditions encountered in the human hip joint.

In this example a test machine ‘8800 Instron System’ has been used.

Results: Following simulation where the device has been treated by 1,000,000 movements of a 100 Kg person, no debris of the material has been observed.

The simulation was continued to 15,000,000 cycles with a load pattern simulating walking. The load varied between 2500 N and about 150 N and the cup rotated in a rotation angle between +15 and −15 degree. The test was regularly stopped with intervals around 1 million cycles, and the specimen was taken out for inspection and photographing.

From 5 to 15 million cycles the thickness of the specimen was measured at each inspection. The wear rate is approximately 3040 μm per 1 million cycles.

### Example 3

Knee Joint

The artificial polymer composite intraarticular implant with improved surface friction modalities should be bipolar with femoral component covering the cartilage area of the medial and lateral femoral condyles, and adjustments and alignment according to cruciate ligaments should be performed.

The thickness is between 2 to 4 mm. The shape mimics the articular surface of conventional total knee arthroplastes.

The tibial component is constructed in same material as above, and the shape and contour follows the meniscus including the central joint area. The implant is connected with an anterior bridge in front of the attachment of the anterior cruciate ligaments attachment on tibia.

Both components are unconstrained to each other and unconstrained to the femoral and tibial parts of the human joint.

The fixation or stability of the implant is dependent on macrostructure of the bony parts and the joint capsule. The stiffness of the implant ensures no roll up phenomenon of each implant component.

### Example 4

Ankle Joint

The artificial polymer ankle joint spacer implant consists of a material close to that mentioned in the example of knee joint.

The implant is monopolar and its extension-corresponds to the cartilage area of the talar bone of the ankle joint. The thickness is between 2 to 4 mm.

The rim of the implant possess a softer rim, holds the implant during loading and flexion and extension.

### Example 5

Shoulder Joint

The artificial polymer shoulder spacer implant consist of a material close to that mentioned in the example of knee joint.

The implant is monopolar and its extension corresponds to the cartilage area of the glenoid cavitate of the shoulder joint plus some extension which may be 0.5 to 3 cm, which articulates with the cartilage area of the head of humerus. The thickness is between 2 to 4 mm. The implant will be excavated superiorly according to the tendon of the long biceps muscle, and it should respect the rotator cuff, accordingly.

The rim of the implant possess a softer rim, holds the implant during loading and flexion and extension, abduction, inner and outer rotation.

1-101. (canceled)

102. A medical device comprising a biocompatible polymeric product with a layered structure comprising:
at least one upper layer of a first polymeric component,
a middle layer at a second polymeric component, and
at least one lower layer of a third polymeric component,
wherein the chain length of the first polymeric component and the third polymeric component is longer than the chain length of the second polymeric component.

103. The medical device according to claim 102, wherein at least one of the first polymeric component, the second polymeric component and the third polymeric component each is independently selected from: the group consisting of polyacrylates, polystyrene, polyethers, polytetrafluoroethylene, polyvinylalcohol, polyethylene, polypropylene, polyethylene oxides and polyvinylpyrrolidone.

104. The medical device according to claim 102, wherein the first polymeric component and the third polymeric component are substantially identical.

105. The medical device according to claim 102, wherein the first and third polymeric components are composed of long polymer fiber, and the second polymeric component is a short chain polymer material.

106. The medical device according to claim 102, wherein the first and third polymeric components are ultra high molecule weight polyethylene (UHMWPE) fiber.

107. The medical device according to claim 102, wherein said at least one upper layer of a first polymeric component and said at least one lower layer of a third polymeric component each comprises a fabric, and wherein the tensile strength of a fiber or strand of the fabric is above 1.0 GPa.

108. The medical device according to claim 102, wherein the polymers of the second polymeric component re short chain polymeric material which may be optionally branched.

109. The medical device according to claim 102, wherein the middle layer is selected from the group consisting of: a core, a film, an inlay and combinations thereof.

110. The medical device according to claim 109 wherein the film is between 0.001 and 5 mm thick.

111. The medical device according to claim 102, wherein the shape of the device is any shape which can be formed by pressing into a mold, and the overall shape of the device is selected from the group consisting of: circular, oval, squared, rectangle, cubed, bowl, cup, crown, cap, basin, hemispherical, and combinations thereof.

112. The medical device according to claim 102, wherein to the polymeric material is attached to a component, said component being polymeric or non-polymeric.

113. The medical devices according to claim 102, wherein the device is supplied with at least one feature selected from the group consisting of apertures, holes, gaps, perforations and hollows.

114. The medical device according to claim 102, wherein the polymeric product is adapted to be used as a medical device for the body of a mammal.

115. The medical device according to claim 102, wherein the polymeric product is adapted not to interfere with intra-articular components when the device is in the body of a human.

116. The medical device according to claim 102, wherein said device is utilized to support, bear, carry, replace or displace any constitution within the human body, which comprises high shape stability and good wear resistance.

117. The medical device according to claim 102, wherein the device at least partially surrounds an intra-articular component.

118. The medical device according to claim 102, wherein the device is a hip endoprosthesis.

119. The medical device according to claim 102, wherein the polymeric product constitutes the surface of a prosthetic device.

120. The medical device according to claim 102, wherein the device is a cartilage substitute.

121. The medical device according to claim 102, wherein the device is a breast prosthesis.

122. The medical device according to claim 102, wherein the device is a stent.

123. The medical device according to claim 102, wherein the device is a catheter.

124. The medical device according to claim 102, wherein the device is a heart valve.

125. A method for producing a biocompatible medical device of a polymeric product, said method comprising:
providing at least three polymer layers, comprising:
at least one upper layer of a first polymeric component,
a middle layer of a second polymeric component, and
at least one lower layer of a third polymeric component,
wherein the chain length of at least one of the at least one first polymeric component and at least one of the at least one third polymeric component is longer than the chain length of the second polymeric component, and
positioning said polymer layers in a sandwich composition, and
shaping the sandwich composition of polymer layers by heating said composition followed by pressing it into a mold, where the heating and pressing processes are conducted in a vacuum, so as to provide the polymeric product in a desired shape.

126. The method according to claim 125, wherein the at least three polymer layers constitute a member which is a core or a film or an inlay, said member having at least one layer of fabric on each side.

127. The method according to claim 126, wherein the member differs in constitution from the fabrics.

128. The method according to claim 126, wherein different layers of fabrics have equal constitutions.

129. The method according to claim 125, wherein the polymer layers are composed of a polymer selected from the group consisting of; polyacrylates, polystyrene, polyethers, polytetrafluorethylene, polyvinylalcohol, polyethylene, polypropylene, polyethylene oxides, polyvinylpyrrolidone, and combinations thereof.

130. The method according to claim 125, wherein the polymer layers are composed of a polymer selected from the group consisting of: polyethylene (FE), polypropylene (PP), polyvinylpyrrolidone (PVP) and combinations thereof.

131. The method according to claim 126, wherein the structure of the fabrics are composed of long polymer fiber, and the member is composed of short chain polymer material.

132. The method according to claim 126, wherein the first and third polymeric components comprise long polymer fiber which is ultra high molecular weight polyethylene.

133. The method according to claim 126, wherein the fabric has high tensile strength and high wear resistance.

134. The method according to claim 125, wherein the second polymeric component comprises short chain polymer material which is branched.

135. The method according to claim 126, wherein the member is a film which is between 0.001 and 5 mm thick.

136. The method according to claim 126, wherein the member is a core which is between 0.1 and 30 mm thick.

137. The method according to claim 125, wherein the heating is at a temperature of between 80 and 250 degrees Celsius.

138. The method according to claim 125, wherein the vacuum is below 800 mbar.

139. The method according to claim 125, wherein the shaped product is treated by radiation, to further crosslink the polymers and thereby improving the strength of the product.

140. The method according to claim 125, wherein the shaped product is further subjected to annealing.

141. The method according to claim 140, wherein the annealed polymeric product is subjected to surface coating, where the product is coated by polyvinylpyrrolidone (PVP) by plasma polymerization.

142, The method according to claim 125, wherein the thickness of the polymeric product is between 0.001 and 40 cm thick.
